(19) United States
(12) Patent Application Publication
(10) Pub. No.: US 2023/0145361 A1
(43) Pub. Date: May 11, 2023

(54) METHOD, COMPRISING BACTERIOPHAGES, FOR REDUCING THE POPULATION OF AT LEAST ONE ADIPOGENIC BACTERIAL SPECIES, AND BACTERIOPHAGES AND THE USE THEREOF

(71) Applicant: OXANA KARPF, Saarbruecken (DE)

(72) Inventor: OXANA KARPF, Saarbruecken (DE)

(21) Appl. No.: 17/795,327

(22) PCT Filed: Jan. 20, 2021
(86) PCT No.: PCT/EP2021/051212

§ 371 Date: Jul. 26, 2022

(30) Foreign Application Priority Data
Jan. 27, 2020 (DE) 10 2020 101 859.2

(51) Int. Cl. C12N 15/86; A61K 35/76; C12N 7/00; C12N 15/11

(52) U.S. Cl. CPC C12N 15/86; A61K 35/76; C12N 7/00; C12N 15/11; C12N 2795/00032; C12N 2795/00043

(57) ABSTRACT

A method for reducing the population of at least one adipogenic bacteria species, the method comprising the following steps: a) providing a biologic sample which comprises at least one adipogenic bacteria species; and b) providing bacteriophages of at least one bacteriophage species which are specific to at least one adipogenic bacteria species and comprise at least one nucleic acid functionally bound to a promoter and/or to a regulatory element; and c) exposing the biologic sample to and incubating it with the bacteriophages, incubation taking place until the population of the adiopogenic bacteria species has been reduced by at least 70%; as well as relating to bacteriophages and their use.

This application represents the national stage entry of PCT International Patent Application No. PCT/EP2021/051212 filed on Jan. 20, 2021 and claims priority to German Patent Application No. 10 2020 101 859.2 filed Jan. 27, 2020. The contents of each of these applications are hereby incorporated by reference as if set forth in their entirety herein.

The disclosure relates to a method for reducing the population of at least one adipogenic bacteria species as well as relating to bacteriophages and their use according to the preamble of the independent claims.

The fact that the interaction between bacteria and their host, i.e., humans and/or animals, can have both positive and negative effects is sufficiently known. On the one hand, bacteria can be symbionts or commensals for humans or animals and can thereby serve or co-exist with them without damaging them. On the other hand, a number of bacteria species is known which can attack and damage the human or animal organism as pathologic organisms or parasites and are suspected of causing and/or benefiting a series of illnesses and associated after-effects, such as the emergence of overweight or excessive weight gain. A damage of this sort can lead to the death of the organism.

To combat a bacterial colonization or bacterial infection, a number of chemical substances have become established as antibiotics which are currently considered the conventional treatment method for humans and animals.

These kinds of substances are characterized by a wide spectrum of activity as they target molecular structures or rather molecules of the bacteria which are found in a number of bacteria species. This permits reducing or eliminating a large range of bacteria by means of antibiotics. A disadvantage of this effect is that the commensal or symbiotic bacteria previously mentioned are also killed by the antibiotic use which can lead to secondary damage to humans, animals and/or the environment. As a consequence of damage to commensal and/or symbiotic bacteria, for example, the balance of the microbiome can shift in favor of pathogenic microorganisms (e.g., bacteria and/or yeasts), an effect which, for example, can lead to a state lasting for several months not uncommonly connected to severe consequences.

Moreover, a possible interaction between chemical substances used as antibiotics and physiological metabolic processes are common knowledge. In this instance, for example, it is known that certain antibiotics have an anabolic effect which is taken advantage of in agriculture. However, it is seen as disadvantageous that the antibiotics used in agriculture are ingested by the human and/or animal organism via the food chain and can lead to fattening effects in these organisms, such as growth acceleration and/or hormonal development. Other forms of incompatibility of the chemical substances which are used as antibiotics are also described. These include, among other things, the emergence of allergies to precisely these chemical substances and the effects of an allergic reaction resulting therefrom to the point of allergic shocks and/or the development of various (food) intolerances. Moreover, in the case of the failure of the antibiotic treatment, repeated treatment with the same medicinal active ingredient is not possible, meaning treatment is commonly switched to a different antibiotic active ingredient group which has an even wider spectrum of activity, consequently leading to an even more profound imbalance of the microbiota, including the emergence of multi-resistant bacteria. Indeed, a specific wait period must be maintained between two antibiotic treatments which in turn, however, can lead to a spreading of the pathologic bacteria. In this case as well as in the case of prematurely terminating the use of antibiotics after treatment has been presumed to be successful, the bacteria which have been exposed to the chemical substance without being killed can develop resistance mechanisms and what is known as resistances to antibiotics. If a resistance arises, then this specific bacterium becomes immune to the antibiotic treatment with the corresponding chemical substance. A treatment or therapy can no longer be successful in this case. This can also come to pass when residual amounts of an antibiotic or in fact its degradation product contaminate potable water and/or the environment and consequently when the bacteria in the environment are exposed to this substance. Not least, it is more difficult for chemical substances used as antibiotics to penetrate bacterially formed biofilms, as the biofilms are characterized by a particularly sugary and thus polar surface which several known antibiotics cannot penetrate.

Overweight, adiposity and/or excessive weight gain can be referred to as a global epidemic by now and are seen as problematic, in particular with regards to physical and mental well-being. These mental and/or physical afflictions can come to pass as medical consequences stemming from overweight, adiposity and/or excessive weight gain, as well as be facilitated owing to increased risks, such as disorders of the metabolism, cardiovascular system, the hormonal balance, the musculoskeletal system or other organ systems. These include disorders or medical conditions such as arteriosclerosis, diabetes, arthralgia, including but not limited to in the spine, hip and knee, indigestion and cancer, such as breast cancer, bowel cancer and pancreatic cancer. Weight loss in the case of overweight and/or adiposity or stabilizing the optimum weight, however, is only possible with great difficulty for many of the affected persons, since our modern lifestyle and/or a familial predisposition make weight reduction difficult for those afflicted with overweight, adiposity and/or excessive weight gain. However, it is this weight reduction that is imperative for overweight persons or persons suffering from severe weight gain, as in particular overweight, adiposity and/or excessive weight gain can cause complications in treatments for a number of illnesses or delay or prevent the success of a treatment. Furthermore, excessive fattening of tissue and/or organs in overweight persons can disrupt the functionality of the organs or even cause organ failure. Further illnesses, to which overweight commonly contributes to at least the emergence or causes complications during the treatment of these illnesses, affect the medical areas of oncology, immunology, gastroenterology, endocrinology, psychiatry, psychosomatics, orthopedics, pediatrics, surgery, urology and others.

Overweight can cause or contribute to illnesses and/or medical conditions or make their treatment more difficult, these illnesses and/or medical conditions being, for example, metabolic syndrome, diabetes mellitus type 2, gall bladder diseases, chronic enteritis, hyperchlorhydria, hypertension, lipometabolic disorders, respiratory difficulties, sleep apnea, coronary heart disease, arthrosis, gout, cancer, such as uterine cancer, breast cancer, cervical cancer, intestinal cancer, prostate cancer and pancreatic cancer, sex hormone disorders, reduced libido, joint and back pain, increased risk of thromboses and embolisms, increased risk during surgeries and narcoses, psychosocial problems and the resulting limitations in life quality, for example resulting from depression, reduced self-worth, for example caused by the perception of being appreciated less by the environment.

The emergence of overweight can also be attributed to bacteria within the intestinal flora. It is thus conceivable that a reduction in the population of the respective bacteria within the intestinal flora prevents or at least limits overweight or excessive weight gain from occurring. A disadvantage here is that conventional antibiotic treatment not only eliminates the targeted bacteria conducive to overweight but also eliminates commensal bacteria which positively affects the host organism, i.e., the human and/or animal organism, and benefits its digestive process. This would also mean further increased circulation of antibiotics which can boost resistances. In other respects, using antibiotics to reduce weight is generally not advisable, as the antibiotics would have to be taken for an extended period of time (e.g., for several months), which would mean excessive damage to the commensal flora and an extended occurrence of side effects related to the antibiotics. A treatment consisting of antibiotics only is thus not advantageous for the treatment and/or therapy of overweight or excessive weight gain.

As a result of the described disadvantages of the conventional treatment with chemical substances used as antibiotics and for the general conventional fight against bacteria populations, in particular for the treatment or therapy of overweight, adiposity and/or excessive weight gain, there is large and continuously increasing demand for modulating, fighting and/or eliminating at least one adipogenic bacteria species in a simple, effective, compatible, medically safe, inexpensive, targeted and specific manner with little side effects. Simultaneously, it is imperative for the treatment to be successful long-term. It is therefore the object of the disclosure to provide bacteriophages and a method for reducing the population of at least one adipogenic bacteria species which in order to prevent the disadvantages of the state of the art as described above and is characterized by high efficacy, specificity, compatibility and/or reliability and by simple, time and cost-efficient use with few flaws.

This object is attained in a surprisingly simple but effective manner by a method and bacteriophages according to the teachings of the independent claims.

According to the disclosure, a method for reducing the population of at least one adipogenic bacteria species is proposed, which comprises the following steps:
a) providing a biologic sample which comprises bacteria of at least one adipogenic bacteria species; and
b) providing bacteriophages of at least one bacteriophage species which are specific to at least one adipogenic bacteria species and which comprise at least one nucleic acid functionally bound to a promoter and/or to a regulatory element, the nucleic acid being chosen from among the group of:
   i. a nucleic acid sequence which codes for at least one antibacterial nucleic acid molecule; and
   ii. a nucleic acid sequence which codes a nucleic acid molecule, at least 50% of which is identical with the nucleic acid molecule coded by the nucleic acid sequences coded in i.; and
   iii. a nucleic acid sequence which codes for at least one antibacterial polypeptide; and
   iv. a nucleic acid sequence which codes a polypeptide, at least 50% of which is identical with a polypeptide coded by the nucleic acid sequences in iii.; and
   v. a nucleic acid sequence for a fragment of a nucleic acid from i, ii, iii, iv, the fragment coding a nucleic acid molecule or a polypeptide; and
c) exposing the biologic sample to and incubating it with the bacteriophages, incubation taking place until the population of the adiopogenic bacteria species has been reduced by at least 70%.

The method according to the disclosure is based on the fundamental idea that by using bacteriophages specific to the at least one adipogenic bacteria species, it is possible to reliably, quickly, easily, specifically and persistently reduce the population of this at least one adipogenic bacteria species. In doing so, it has proven that advantageously other organisms, such as yeasts, bacteria, viruses or the like, and/or cells of multicellular tissue and/or organisms remain unaffected owing to the simultaneous host dependency, meaning no damage, impairments and/or disruptions thereof arise. Owing to the host dependency of the bacteriophages, the method according to the disclosure can be repeated at any time, without any resistances arising. As a person skilled in the art is familiar with, a reciprocal host dependency exists if there is no reduction of a different bacteria species within the biologic sample for which the bacteriophages is not specific when the respective adipogenic bacteria species is no longer present within the biologic sample. This also pertains to organs and/or tissue of the host organism, i.e., of the human and/or animal organism.

Within the scope of the disclosure, the discovery has been made that for reducing the population of at least one bacteria species, it is sufficient to provide a biologic sample which comprises bacteria of at least one adipogenic bacteria species and to incubate this bacteria species with the administered bacteriophages of at least one bacteriophage species which is specific to the at least one adipogenic bacteria species.

The term “population” is known to the person skilled in the art and generally pertains to the totality of all individuals of the same species which exist in a certain areal and/or in a certain area of the environment. Within the scope of the disclosure, the population pertains to the totality of the bacteria of at least one adipogenic bacteria species. The terms “bacterium” and “bacteria” are known to the person skilled in the art as interchangeable synonyms and refer to the representatives of one or more bacteria species.

The term “adipogenic bacteria species” is known to the person skilled in the art and pertains to at least one species of bacteria, preferably 2, 3, 4, 5, 6, 7, 8, 9, 10, 11, 12, 13, 14, 15, 16, 17, 18, 19, 20 or more bacteria species which directly or indirectly impact a human and/or animal organism in such a manner owing to their mode of living, e.g., via their molecular metabolism and/or their interaction with other bacteria species, yeasts and/or cells, that obesity, adiposity, overweight and/or weight gain is initiated, favored and/or increased by the structure, storing and/or decreased reduction of fat tissue, preferably of white adipose tissue. A person skilled in the art knows of a direct or indirect impact on the organism. Thus, they can, for example, directly affect the organism by destroying tissue of the organism, or they can indirectly affect the metabolic process or other processes in the organism, whereby, among other things, weight gain and/or a non-reduction of overweight can be initiated and/or favored. Furthermore, the person skilled in the art also understands that “adipogenic” in this context can also mean the corresponding bacteria species itself may not directly cause an adipogenic effect but that, however, it can be opposed to the growth, mode of life and/or survival of an anti-adipogenic bacteria species and in this manner has an adipogenic effect on the organism. Within the scope of the disclosure, it is conceivable that the adipogenic bacteria species can be present within the gastrointestinal tract of the human and/or animal organism, such as in the stomach and/or in the intestines.

The adipogenic bacteria species is chosen from but not limited to: Acinetobacter, Actinobacteria, Actinomyces, Bacilli, Bacteriodes, Bartonella, Bordetella, Borrelii, Brucella, Citrobacter, Campylobacter, Chlamydia, Clostridia, Corynebacteria, Desulfovibrio, Ehrlichia, Enterobacter, Enterobacteriaceae, Enterococcus, Erysipelotrichia, Escherichia, Faecalibacterium, Firmicutes, Francisella, Helicobacter, Hemophili, Klebsiella, Lactobacillus, Legionella, Leptospira, Listeria, Methanobrevibacter, Moraxella, Mycobacterium, Mycoplasmi, Neisserii, Nocardia, Oscillobacter, Paeruginosa, Prevotellaceae, Propionibacterium, Proteus, Pseudomonas, Rickettsia, Ruminococci, Salmonella, Shigella, Spirrillum, Spirochetes, Staphylococci, Stenotrophomonas, Streptobacilli, Streptococci, Treponema, Vibrio, Yersenia, Bifidobacterium, Blautia, Bulleidia, Coprococcus, Dialister, Eubacterium, Lachnospiraceae, Oribacterium, Roseburia, Christensenellaceae, Erwinia, Flavonifractor, Oscillospira, Phascolarctobacterium, Prevotella, Succinivibrio, Ruminococcus and/or Veillonella.

Furthermore, the adipogenic bacteria species is chosen from but not limited to: Acinetobacter baumannii, Bacillus cereus, Bacillus anthracis, Bacillus subtilis, Bacteriodes thetaiotaomicron, Bacteriodes vulgatus, Bartonella henselae, Bordetella pertussis, Borrelia recurrentis, Borrelia hermsii, Borrelia turicatue, Borrelia burgdorferi, Campylobacter jejuni, Citrobacter fruendii, Chlamydia psittaci, Chlamydia trachomatis, Chlamydia pneumoniae, Clostridium botulinum, Clostridium difficle, Clostridium tetani, Clostridium perfringens, Clostridium ramosum, Clostridium novyi, Clostridium septicum, Clostridium leptum, Corynebacteria diptheriae, Desulfovibrio piger, Ehrlichia chaffeensis, Enterococcus faecalis, Escherichia coli (for example EHEC, EIEC, ETEC), Faecalibacterium prausnitzii, Francisella tularensis, Helicobacter pylori, Hemophilus influenzae, Hemophilus parainfluenzae, Hemophilus aegyptus, Klebsiella pneumoniae, Lactobacillus reuteri, Legionella pneumophila, Leptospirex hemoragia, Leptospira icterohemorrhagiae, Listeria monocytogenes, Methanobrevibacter smithii, Moraxella catarrhalis, Mycobacterium tuberculosis, Mycobacterium leprae, Mycobacterium asiaticum, Mycobacterium intracellulare, Mycobacterium avium-intracellulars, Myobacterium johnei, Mycobacterium avium, Mycobacterium smegmatis, Mycoplasma pneumoniae, Mycoplasma hominis, Neisseria meningitidis, Neisseria gonorrhea, Rickettsia prowozekii, Rickettsia rickettsia, Rickettsia akari, Propionibacterium acnes, Pseudomonas aeruginosa, Pseudomonas syringae, Salmonella typhimurium, Salmonella typhi, Salmonella paratyphi, Salmonella schottmulleri, Salmonella hirshieldii, Shigella dysenteriae, Spirrillum minus, Staphylococcus aureus, Staphylococcus epidermidis, Stenotrophomonas maltophilia, Streptobacillus moniliformis, Streptococcus pneumoniae, Streptococcus mutans, Streptococcus oralis, Streptococcus parasanguis, Streptococcus pyogenes, Streptococcus viridans, Streptococcus coelicolor, Streptococcus agalactiae, Streptococcus bovis, Treponema pallidum, Treponema pertainue, Treponema carateum, Vibrio cholera, Vibrio parahaemolyticus, Yersenia pestis, Yersinia enterocolitica, Bacteroides fragilis, Blautia hydrogenotorophica, Coprococcus catus, Eubacterium ventriosum, Bacteroides faecichinchillae, Bifidobacterium animalis, Blautia wexlerae, Clostridium bolteae, Flavonifractor plautii, Lactobacillus gasseri, Ruminococcus bromii and/or Ruminococcus obeum. In this case, it is presumed that the respective bacteria species has a known and/or suspected adipogenic effect on a human and/or animal organism.

In the first step of the method according to the disclosure, it is necessary to provide a biologic sample comprising bacteria of at least one adipogenic bacteria species. The term “biologic sample” pertains to a material of a human and/or animal organism and/or to a material which has been exposed to a human and/or animal organism for at least a short period of time. In this context, it has been discovered to be essential for the biologic sample to comprise bacteria of at least one adipogenic bacteria species, preferably 2, 3, 4, 5, 6, 7, 8, 9, 10, 11, 12, 13, 14, 15, 16, 17, 18, 19, 20 or more bacteria species. Samples of this kind are known to a person skilled in the art, such as cell or nucleus-containing (DNA and/or RNA-containing) samples of, among other things, saliva, urine, blood, feces, sweat, cellular tissue, organ puncture, organs, parts of organs, whole organisms (including dead/necrotic), soil samples, water samples or similar samples.

In the next step of the method according to the disclosure, it is necessary to provide bacteriophages of at least one bacteriophage species which is specific to at least one adipogenic bacteria species. The term “bacteriophage” or “bacteriophage species” is understood by a person skilled in the art and pertains to infectious organic structures which can spread outside of cells and multiply within a cell. The cells serve as a host and are essentially bacteria in the case of bacteriophages, which have a pronounced host specificity, i.e., are specific to at least one bacteria species and thus only use the bacteria for which they are specific to spread and multiply.

It is essential that the bacteriophages used for the method according to the disclosure comprise at least one nucleic acid functionally bound with a promoter and/or a regulatory element or have a promoter and/or a regulatory element in combination with a nucleic acid, the nucleic acid coding either for an antibacterial nucleic acid molecule, a bacterial polypeptide and/or a fragment thereof. Preferably, the bacteriophages comprise a nucleic acid having at least 2, 3, 4, 5, 6, 7, 8, 9, 10, 11, 12, 13, 14, 15, 16, 17, 18, 19, 20 or more promoters and/or regulator elements. The nucleic acid sequences which code for promoters or regulatory sequences are sufficiently known to the person skilled in the art.

A person skilled in the art knows that the terms “nucleic acid”, “genes”, “DNA”, “RNA”, “mRNA”, “cRNA”, “miRNA” and the compounds “nucleic acid sequence” and “nucleic acid molecule” are used as interchangeable synonyms for one another to describe deoxyribonucleotides and ribonucleotides and polymers thereof, either single or double-stranded.

The terms “promoter” and “regulatory sequence” and/or “regulatory element” are also familiar to the person skilled in the art and pertain to nucleic acid sequences of the DNA which enable and/or increase the expression, i.e., the transcription of a gene to mRNA. In this case, enhancer elements, i.e., binding locations for growth factors, hormones, oncogenes and/or the like, can be regulatory elements.

A person skilled in the art is further familiar with the terms “polypeptide”, “peptide”, “amino acids”, and “protein” being used as interchangeable synonyms for each other to refer to a polymer of amino acid residue. Naturally occurring amino acids are those which are coded by the genetic code as well as those amino acids which can be modified at a later time. Polypeptides can be present as a residual substance, as a transport and/or signal molecule, as a structure molecule, as a protective and/or defensive molecule and/or as a metabolically active molecule and can implement different functions within and/or without a cell and/or in the organism. Polypeptides can, for example, have an antibacterial affect and thus counteract the mode of living of bacteria owing to their properties or functions.

Within the scope of the method according to the disclosure, at least one antibacterial nucleic acid molecule, at least one antibacterial polypeptide and/or at least a fragment thereof is provided; preferably the bacteriophages can code 2, 3, 4, 5, 6, 7, 8, 9, 10, 11, 12, 13, 14, 15, 16, 17, 18, 19, 20 or more antibacterial nucleic acid molecules, antibacterial polypeptides and/or fragments thereof. Antibacterial nucleic acid molecules can derive from the area of RNA species and be functional mRNA or miRNA, for example. Polypeptides can be present as simple linear polypeptides or also be folded structure proteins. Other possible proteins can also be molecules which act as toxins, growth-inhibiting substances and enzymes or molecules for inhibiting cell division. Possible nucleic acid molecules, for example RNA molecules, are those which, for example, inhibit metabolic processes, impede cell division or promote dysfunctions of the biologic processes within the bacteria.

Within the scope of the method according to the disclosure, a nucleic acid sequence is conceivable which codes a nucleic acid molecule, a polypeptide and/or a fragment thereof and at least 50% of which is identical with the antibacterial nucleic acid sequence mentioned above or with an antibacterial polypeptide. Preferably, a nucleic acid sequence is also conceivable at least 55%, at least 60%, at least 65%, at least 70%, at least 75%, at least 80%, at least 85%, at least 90%, at least 95%, at least 96%, at least 97%, at least 98%, at least 99% or 100% of which is identical with the antibacterial nucleic acid sequence mentioned above or with the antibacterial polypeptide. In this context, the nucleic acid sequence is a naturally occurring nucleic acid sequence or a non-naturally occurring nucleic acid sequence which is already present in bacteriophages or which was introduced to the respective bacteriophages via molecular-biological methods, for example. For instance, these molecular-biological methods can refer to in vivo and/or in vitro recombination, gene transfer, CRISPR/Cas, TALEN or the like.

The bacteriophages can belong to the order Caudovirales, Ligamenviralis or the like. Preferably, the bacteriophages belong to the family Ackermanviridae, Ampullaviridae, Bicaudaviridae, Clavaviridae, Corticoviridae, Fuselloviridae, Globuloviridae, Guttaviridae, Lipothrixviridae, Myoviridae, Plasmaviridae, Podoviridae, Portogloboviridae, Rudiviridae, Salterprovirus, Sphaerolipoviridae, Siphoviridae, Tectiviridae, Tristomaviridae, Turriviridae, Inoviridae, Microviridae, Spiraviridae, Pleolipoviridae, Cystoviridae or Leviviridae. Within the scope of the disclosure, it is conceivable that bacteriophages of at least one, preferably 2, 3, 4, 5, 6, 7, 8, 9, 10, 11, 12, 13, 14, 15, 16, 17, 18, 19, 20 or more, bacteriophage species are provided. Furthermore, it is conceivable that the bacteriophages of a bacteriophage species are specific to at least one, and preferably 2, 3, 4, 5, 6, 7, 8, 9, 10, 11, 12, 13, 14, 15, 16, 17, 18, 19, 20 or more, adipogenic bacteria species. A targeted selection of a suitable bacteriophage for the corresponding bacteria species is known to the person skilled in the art and can be easily selected from databanks, for example.

In the next step of the method according to the disclosure, it is necessary to expose the biologic sample to the bacteriophages and to incubate them together. The biologic sample and the bacteriophages can be exposed to each other by dribbling, injecting, placing on top of each other and/or via a different method known to the person skilled in the art.

The term “incubating” associated with the method according to the disclosure pertains to a method step during which the biologic sample is incubated with the bacteriophages over a specific time period; preferably under the corresponding circumstances known to the person skilled in the art, a reproduction cycle of the bacteriophages within the biologic sample comprising the bacteria of the at least one bacteria species specific to the bacteriophages is the result. Besides incubating a biologic sample with bacteriophages, this method step also comprises monitoring the biologic sample with bacteriophages until the population of the at least one bacteria species has been reduced by at least 70%. The implementation of the monitoring is sufficiently known to the person skilled in the art and can take place, for example, because of a predetermined time period or be based on the skilled person's experience, for example by observing the sample. Alternatively, a colored or other visually perceivable indication can be intended which signals when the population of the bacteria species has been reduced by at least 70%. This can take place, for example, when the desired bacteria density has been attained or by means of a chemical and/or biochemical indicator, such as by means of a coloration, a color change of the coloration or color loss. The incubating and monitoring steps can take place reciprocally until the bacteria species has been reduced by at least 70%.

Within the scope of the disclosure, it has proven advantageous when the population of the bacteria species is reduced by at least 70%. Preferably, the population can be reduced by more than 70%, for example at least 75%, at least 80%, at least 85%, at least 90%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, at least 99.9% or by 100%; a reduction of at least 70% of the population of the bacteria species is deemed considerable, while a reduction by 95% of the population of the bacteria species is deemed significant. The person skilled in the art knows that a reduction is rarely 100%, even if the use of suitable bacteriophages in a suitable biologic sample may bring about this result.

A reduction of the population of the bacteria species by 70% to 100% moreover allows the person skilled in the art to recognize the success of the method which accounts for a sufficient antibacterial effect and thus a sufficient reduction of the adipogenic bacteria species within the biologic sample. The reduction of the population of the bacteria species can be used as a metric for the efficacy or success of a weight-reducing treatment based on the use of respective bacteriophages. Alternatively, a reduction of a bacteria species can be targeted which opposes a life or mode of life of an anti-adipogenic bacteria species. Consequently, an anti-adipogenic bacteria species can continue to persist or grow while the bacteria species counteracting the anti-adipogenic bacteria species is reduced even though the anti-adipogenic bacteria is specific to the bacteriophages. In this context, it is presumed that the result of a growth of a population of an anti-adipogenic bacteria species can have the same effect on an organism as the reduction of a population of an adipogenic bacteria species.

By means of the method according to the disclosure, it is thus possible to reduce the population of at least one adipogenic bacteria species specifically, effectively, compatibly, medically safely, permanently and persistently and with little side effects within a biologic sample, without damaging and/or affecting bacteria or other cells, tissue and/or organisms within the same biologic sample for which the bacteriophages does not have any specificity. In this manner, in contrast to the use of antibiotics, damage to other bacteria species, such as yeasts, tissues and/or organs in the human and/or animal organism is entirely circumvented. The method according to the disclosure cannot only be reliably and cost-effectively implemented but also quickly and easily, as the few method steps can be implemented in a standard manner and a high success rate is achieved by a considerable (more than 70%) reduction or a significant (more than 95%) reduction of the bacteria species. The method moreover can be implemented using simple means and solely requires devices familiar to the person skilled in the art for providing the biologic sample and the bacteriophages, for exposing and incubating the same and for evaluating the reduction of the respective bacteria species within the population.

Advantageous further embodiments of the disclosure, which can be realized individually or in combination, are represented in the dependent claims.

With a further embodiment of the disclosure, it is conceivable that subsequently to step c), the reduction of the population of the adipogenic bacteria species is evaluated. The term “evaluating” is perspicuous to the person skilled in the art and pertains to the analysis, evaluation and/or assessment of step c). Since a reduction of the population of the bacteria species of at least 70% has been monitored, this method step serves in particular for verifying the achieved reduction as well as to recording the result by preparing written notes, photographs and video recordings, acquiring and documenting data regarding enumerations, density measurements, growth determination or similar methods and can be used for creating statistics regarding the repeated implementation of the method according to the disclosure. In this step, the vitality and/or viability of the bacteria can be established which remain in the biologic sample and were not or were not sufficiently killed by bacteriophages. In this context, an assessment of the vitality of bacteria is possible for which the respective bacteriophages are specific. Common methods for this purpose are also known to the person skilled in the art.

It is further conceivable that the nucleic acid has two or more promoters and/or regulatory elements in step b), preferably 2, 3, 4, 5, 6, 7, 8, 9, 10, 11, 12, 13, 14, 15, 16, 17, 18, 19, 20 or more promoters and/or regulatory elements being present. It is essential in this context that a particularly strong activation of the expression of the nucleic acid and thus an increased and efficient production of the antibacterial nucleic acid, the antibacterial nucleic acid molecules, the antibacterial polypeptide and/or the fragment thereof can be achieved. A bacteriophage having such a nucleic acid can contribute to the reduction of the population of the bacteria species particularly efficiently and, for example, considerably accelerate and/or considerably shorten step c) (incubation) of the method according to disclosure.

In yet another further embodiment, it is conceivable that the nucleic acid and nucleic acid sequences codes for two or more nucleic acid molecules, polypeptides and/or fragments thereof, the nucleic acid preferably coding for 2, 3, 4, 5, 6, 7, 8, 9, 10, 11, 12, 13, 14, 15, 16, 17, 18, 19, 20 or more nucleic acid molecules, polypeptides and/or fragments thereof. These nucleic acid molecules, polypeptides and/or fragments thereof can have an antibacterial effect for one or more bacteria species; it is preferred that bacteriophages having such a nucleic acid can infect several bacteria species. Furthermore, it can be intended that the nucleic acid sequences are specific to killing one bacteria species. For this purpose, the method can be sped up and a considerable or significant reduction of the population of the bacteria species can be achieved within the biologic sample. This increases efficiency of the method while also reducing the duration and/or the costs when implementing the method.

In another embodiment of the method, it is conceivable that the adipogenic bacteria species is anaerobic and/or aerobic, and the bacteria species is attributable to the strain Actinobacteria, Bacteroidetes, Firmicutes or Proteobacteria.

The terms “aerobic” and “anaerobic” are sufficiently known to the person skilled in the art and describe a preferred mode of living of the respective organism. In this context, an “aerobic” mode of living is understood to mean that the organism depends on aerial oxygen or rather on oxygen in the air for survival. In contrast, “anaerobic” describes a mode of living which survives without oxygen, meaning the respective organism can maintain its molecular metabolism without oxygen. A differentiation is made between “obligatively”, “tolerably” and “facultatively” aerobic and/or anaerobic organisms, the person skilled in the art understanding that “obligative” describes an exclusively aerobic or anaerobic mode of living which precludes the respective other mode of living. “Tolerant”, however, means that the organisms live aerobically or anaerobically but are able to tolerate the opposite oxygen conditions, at least for a certain period of time. Finally, the term “facultative” means that anaerobic organisms, for example, generally maintain a corresponding metabolic process but are able to switch to an aerobic metabolic process when presented with oxygen and vice versa. Thus, bacteria species can have an exclusively aerobic or aerobic mode of living or a combination thereof.

It is presumed that the definition and/or embodiments of the terms mentioned above apply to all aspects described in the following description, provided it has not been indicated otherwise.

According to the disclosure, a kit for implementing the method according to the disclosure is further proposed, the kit comprising
a) a container comprising bacteriophages of at least one bacteriophage species, which are specific to at least one adipogenic bacteria species and comprise at least one nucleic acid functionally bound to a promoter and/or a regulatory element; and
b) instructions for implementing the method.

The term “kit”, as used here, pertains to a collection of the previously mentioned components (kit-of-parts), which are provided preferably separately or within an individual container.

The term “instructions for implementing the method” pertains to a numerical, written and/or graphic representation of the description which allows implementing the method aim in a simplified manner. The description can preferably be available in the form of a handbook or be provided via a computer program, such as an application. Furthermore, it is conceivable that the computer program has an implemented algorithm which is capable of implementing the identification, comparison and/or result thereof, which is referenced in the method of the disclosure at hand. The computer program can be made available on a data storage medium or a device such as an optical storage medium (for example a compact disc) or directly on a computer or a data processing device. In addition, the instructions can preferably comprise standards for usage amounts as they are known to the person skilled in the art. Moreover, the instructions comprise prerequisites for the storage and disposal of the container and the biologic sample. What is more, the instructions for implementing the method can contain tables, registers, databanks and excerpts of the same about the adipogenic, anti-adipogenic and/or other bacteria species and bacteriophage species known to the person skilled in the art; with regard to the bacteriophage species, information on their specificity and/or efficiency can be listed as well.

According to the disclosure, a bacteriophage is further proposed which comprises at least one nucleic acid functionally bound to a promoter and/or to a regulatory element, the bacteriophage being specific to the at least one adipogenic bacteria species and the nucleic acid being chosen from among a group of:
a) a nucleic acid sequence which codes for at least one antibacterial nucleic acid molecule; and
b) a nucleic acid sequence which codes a nucleic acid molecule, at least 50% of which is identical with the nucleic acid molecule coded by the nucleic acid sequences from a); and
c) a nucleic acid sequence which codes for at least one antibacterial polypeptide; and
d) a nucleic acid sequence which codes a polypeptide, at least 50% of which is identical with a polypeptide coded by the nucleic acid sequence from c); and
e) a nucleic acid sequence for a fragment of a nucleic acid from a), b), c) or d), the fragment coding a nucleic acid molecule or a polypeptide.

The bacteriophage according to the disclosure is described in detail above, the nucleic acid sequence being a naturally occurring nucleic acid sequence or a non-naturally occurring nucleic acid which is already present in the bacteriophage or has been introduced in the bacteriophage via molecular-biological methods. For instance, these molecular-biological methods can pertain to in vivo and/or in vitro recombination, gene transfer, CRISPR/Cas, TALEN or the like, as known to a person skilled in the art.

In a further embodiment, it is conceivable that the nucleic acid has two or more promoters and/or regulatory elements. It is essential that a particularly strong activation of the expressions of the nucleic acid and thus a particularly increased and efficient production of the antibacterial nucleic acid, the antibacterial nucleic acid molecules, the antibacterial polypeptides and/or the fragment thereof can be achieved. The bacteriophage having such a nucleic acid can contribute particularly efficiently to reducing the population of the adipogenic bacteria species or to reducing a bacteria species which opposes the mode of living of an anti-adipogenic bacteria species.

Furthermore, it is conceivable that the nucleic acid codes nucleic acid sequences for two or more nucleic acid molecules, polypeptides and/or fragments thereof. It is preferred in this case that the bacteriophage can infect several bacteria species using such a nucleic acid.

In yet another embodiment, it is conceivable that the adipogenic bacteria species is anaerobic and/or aerobic and the bacteria species is attributable to the strain Actionbacteria, Bacteroidetes, Firmicutes or Proteobacteria.

Furthermore according to the disclosure, bacteriophages and/or a pharmaceutic composition comprising bacteriophages and at least one other component are proposed for use in medication to change intestinal flora, in medication for achieving and/or maintaining weight reduction, in medication to prevent weight gain and/or in medication for treating overweight, obesity, metabolic disorder, cardiovascular disease, bacterial infection, metabolic disease, metabolic syndrome and/or cancer.

The term “pharmaceutic composition” used here pertains to a mixture of bacteriophages, which have been described in further detail above, and at least one other component. Preferably, such other components can be stabilizers, wetting agents, pharmaceutical carriers, pharmaceutically acceptable carriers, diluents, pharmaceutically acceptable diluents, additional pharmaceutical active ingredients, release agents and the like. Preferred diluents are water, alcohols, physiological saline solutions, buffers, such as phosphate-buffered saline solutions, syrup, oil, water, emulsions, different types of wetting agents and the like. The carrier must be acceptable in the sense that it is compatible with the other components of the composition and that it is not harmful to the human and/or animal organism. The used pharmaceutical carrier can contain a solid, a gel or a liquid. Examples for solid carriers are lactose, terra alba, sucrose, talcum powder, gelatin, agar, pectin, acacia, magnesium stearate, stearic acid and the like. In a similar manner, the carrier or the diluent can contain a time delay material known well in the technical field, such as glycerol monostearate or glycerol distearate on its own or with a wax. These suitable carriers comprise the carriers mentioned above and other carriers known in the field, as mentioned, for example, in Remington's Pharmaceutical Sciences, Mack Publishing Company, Easton, Pa.; the European Pharmacopeia; the Homeopathic Pharmacopeia of the United States; or the Homeopathic Pharmacopeia (HAB). The pharmaceutically acceptable diluent is chosen such that the biologic activity of the combination is not affected. Examples for such diluents are distilled water, physiological saline solutions, Ringer's solution, dextrose solutions and HANK's solution. Moreover, the pharmaceutical composition can also contain other carriers, additives or non-toxic, non-therapeutic, non-immunogenic stabilizers and the like.

The bacteriophages and/or the pharmaceutical composition are to be adapted to each specific use. Accordingly, it can be presumed that the bacteriophages and/or the pharmaceutical composition can be formulated for a systemic or topical application depending on the intended administration. Preferably, the bacteriophages and/or the pharmaceutical composition are to be formulated for a systemic or local administration. Preferably, an oral application, for example in the form of tablets, solutions or drinking ampules, or a topical administration in gel form or an administration by injection is intended. Depending on the type and mode of acting, the bacteriophages and/or the pharmaceutical composition can, however, be administered in different manners, including dermal, intramuscular, subcutaneous, oral, rectal, retrograde or intravenous administration. In principal, the precise, individually suggested dosage can depend on further parameters well known to specialists of the field. Children can receive a different dose than adults, for example. The medical professional can easily determine whether the dose must be adapted by using different calculation tools at their disposal.

The term “change in the composition of the intestinal flora” describes a process understandable to a person skilled in the art during which at least one population of an adipogenic bacteria species, which is a component of the intestinal flora of the human and/or animal organism, is increased, reduced and/or affected in other manners. Reviewed in isolation, this can be measured by determining the corresponding bacteria number or be determined relative to other bacteria species within the intestinal flora. Suitable methods are common knowledge to the person skilled in the art.

The term “achieving and/or maintaining weight reduction” is understandable to the person skilled in the art and pertains to the short-term reduction of body weight of the human and/or animal organism and/or, preferably, the persisting holding of the reduction of body weight of the human and/or animal organism. The term “preventing weight gain” is also understandable to a person skilled in the art and pertains to the persisting and/or continued holding of reduced body weight of the human and/or animal organism. In this case, it is preferably conceivable that achieving and/or maintaining the weight reduction and preventing weight gain in the human and/or animal organism is achieved by reducing at least one adipogenic bacteria species.

The use of the bacteriophages and/or the pharmaceutical composition in medication can occur, among other things, for treating illnesses in the medical fields of oncology, immunology, infectiology, oral and maxillofacial surgery, otorhinolaryngology, ophthalmology, neurology, gynecology, gastroenterology, endocrinology, psychiatry, psychosomatics, orthopedics, pediatrics, surgery, urology, and/or the like. Possible diagnoses which can indicate use of the bacteriophages and/or the pharmaceutical composition can be, among other things, metabolic syndrome, diabetes mellitus type 2, gall bladder diseases, chronic diseases of the digestive tract, chronic intestinal inflammations, hypochlorhydria, hypertension, lipometabolic disorder, respiratory difficulties, sleep apnea, coronary heart disease, arthrosis, gout, cancer, such as uterine cancer, breast cancer, cervical cancer, pancreatic cancer, liver cancer, stomach cancer, bowel cancer, prostate cancer and gall bladder cancer, sex hormone disorders, reduced libido, joint and back pain, increased risk of thromboses and embolisms, increased risks during surgeries and narcoses, psychosocial problems and the resulting limitations in life quality, for example resulting from depression, reduced self-worth and/or caused by the perception of being appreciated less by the environment. Preferably, the aim in each case is to reduce at least one adipogenic bacteria species and thus leading to achieving and/or maintaining weight reduction and/or preventing weight gain in the human and/or animal organism as the main or secondary success in order to obtain a good, preferably improved, prognostic for the treatment of the respective illness and/or the respective affliction. Conceivable is a use of the bacteriophages and/or the pharmaceutical composition which does not depend on the actual illness. The treatment is deemed successful when at least one adipogenic bacteria species or weight has been reduced and/or when weight gain has been stopped or delayed.

The term “treatment” pertains to each improvement of the human and/or animal organism which occurs in comparison with an untreated human and/or animal organism, this improvement preferably being based on the reduction of at least one adipogenic bacteria species, achieving and/or maintaining weight reduction and/or preventing weight gain in the human and/or animal organism. It is presumed that treatment possibly is not successful in 100% of the human and/or animal organisms to be treated. The term presumes, however, that the treatment is successful for a statistically significant portion of test subjects (for example, a cohort in a cohort study). The person skilled in the art can easily establish whether a portion is statistically significant using different known statistical evaluation instruments, e.g., determining confidence intervals, p-value determination, student's t-test, Mann-Whitney U-test, etc. Preferred confidence intervals are at least 90%, at least 95%, at least 97%, at least 98% or at least 99%. The p-values are preferably 0.05; 0.01; 0.005 or 0.0001.

The term “medication” pertains to bacteriophages and/or a pharmaceutical composition in a therapeutically effective dosage, as described previously. Preferably, the pharmaceutical composition has bacteriophages, at least one pharmaceutically acceptable carrier and/or a diluent. The medication can be formulated for different administration modes which were described in detail above. A therapeutically effective dosage pertains to the amounts required for changing the composition of the intestinal flora, for reducing at least one adipogenic bacteria species, for achieving and/or maintaining weight reduction, for preventing weight gain and/or for treating the illnesses mentioned above. The therapeutic efficacy and toxicity can be determined via pharmaceutical standard methods in cell cultures or test animals, for example ED50 (the dosage which is therapeutically effective in 50% of cases), and LD50 (the dosage which is lethal in 50% in cases). The dosage ratio between therapeutic and toxic effects is the therapeutic index which can be expressed as the LD50/ED50 ratio. The dosage scheme is determined by the practicing doctor and other clinical factors. As known in the medical field, the corresponding dosage depends on many factors known to a medical professional, including, height, body surface, age, the substance to be administered, gender, time and administration mode, general state of health and other medication administered at the same time. Progress can be monitored via a periodic evaluation.

Furthermore, bacteriophages according to the disclosure and/or a pharmaceutical composition comprising bacteriophages and at last one other component are proposed for use in treating overweight, obesity, metabolic disorder, cardiovascular disease, bacterial infection, metabolic disease, metabolic syndrome and/or cancer.

Further according to the disclosure, bacteriophages and/or a pharmaceutical composition comprising bacteriophages and at least one other component are proposed for use in a therapeutic method or in a non-therapeutic method to change the composition of intestinal flora, in a therapeutic method and/or in a non-therapeutic method for achieving and/or maintaining weight loss, in a therapeutic method or in a non-therapeutic method for preventing weight gain and/or in a therapeutic method and/or in a non-therapeutic method for treating overweight, obesity, metabolic disorder, cardiovascular disease, bacterial infection, metabolic disease, metabolic syndrome and/or cancer.

In another embodiment of the disclosure, it is conceivable that the bacterial infection is a bacterial infection of the respiratory paths, the teeth, the mouth, the jaw, the eye, the musculoskeletal system, the blood, the gastrointestinal tract, preferably a bacterial infection of the large intestine, the small intestine, the duodenum, the stomach, the liver, the gall bladder and/or the pancreas, the skin, the cardiovascular system, the hormone balance, the psyche, the immune system, the nervous system, the metabolic system, wounds and/or the urogenital tract.

The term “gastrointestinal tract” is known to the person skilled in the art and pertains to organs in the human and/or animal organism which serve for ingesting, grinding, transporting and/or processing nutrients, with the goal of making the nutrients usable for the human and/or animal organism, the intestine, in particular via the intestinal flora, significantly contributing to the regulation thereof.

The described features can each be realized on their own or in combination with each other. The disclosure is not limited to the exemplary embodiments contained herein

1. A method for reducing the population of at least one adipogenic bacteria species, the method comprising the following steps:
a) providing a biologic sample which comprises bacteria of at least one adipogenic bacteria species; and
b) providing bacteriophages of at least one bacteriophage species which are specific to at least one adipogenic bacteria species and which comprise at least one nucleic acid functionally bound to a promoter and/or to a regulatory element, the nucleic acid being chosen from among the group of:
i. a nucleic acid sequence which codes for at least one antibacterial nucleic acid molecule; and
ii. a nucleic acid sequence which codes a nucleic acid molecule, at least 50% of which is identical with the nucleic acid molecule coded by the nucleic acid sequences coded in i.; and
iii. a nucleic acid sequence which codes for at least one antibacterial polypeptide; and
iv. a nucleic acid sequence which codes a polypeptide, at least 50% of which is identical with a polypeptide coded by the nucleic acid sequences in iii.; and
v. a nucleic acid sequence for a fragment of a nucleic acid from i, ii, iii, iv, the fragment coding a nucleic acid molecule or a polypeptide; and
c) exposing the biologic sample to and incubating it with the bacteriophages, incubation taking place until the population of the adiopogenic bacteria species has been reduced by at least 70%.

2. The method according to claim 1, wherein subsequently to step c), the reduction of the population of the adipogenic bacteria species is evaluated.

3. The method according to claim 1, wherein the nucleic acid has two or more promoters and/or regulatory elements.

4. The method according to claim 1, wherein the nucleic acid codes nucleic acid sequences for two or more nucleic acid molecules, polypeptides and/or fragments of the same.

5. The method according to claim 1, wherein the adipogenic bacteria species is anaerobic and/or aerobic and wherein the bacteria species is attributable to the strain Actinobacteria, Bacteroidetes, Firmicutes or Proteobacteria.

6. A kit for implementing the method according to claim 1 comprising:
a) a container comprising bacteriophages of at least one bacteriophage species, which are specific to at least one adipogenic bacteria species and comprise at least one nucleic acid functionally bound to a promoter and/or a regulatory element; and
b) instructions for implementing the method.

7. A bacteriophage comprising at least one nucleic acid functionally bound to a promoter and/or a regulatory element, the bacteriophage being specific to at least one adipogenic group of bacteria and the nucleic acid being chosen from among the group of:
a) a nucleic acid sequence which codes for at least one antibacterial nucleic acid molecule; and
b) a nucleic acid sequence which codes a nucleic acid molecule, at least 50% of which is identical with the nucleic acid molecule coded by the nucleic acid sequences from a); and
c) a nucleic acid sequence which codes for at least one antibacterial polypeptide; and
d) a nucleic acid sequence which codes a polypeptide, at least 50% of which is identical with a polypeptide coded by the nucleic acid sequence from c); and
e) a nucleic acid sequence for a fragment of a nucleic acid from a), b), c) or d), the fragment coding a nucleic acid molecule or a polypeptide.

8. The bacteriophage according to claim 7, wherein the nucleic acid has two or more promoters and/or regulatory elements.

9. The bacteriophage according to claim 7, wherein the nucleic acid codes nucleic acid sequences for two or more nucleic acid molecules, polypeptides and/or fragments of the same.

10. The bacteriophage according to claim 7, wherein the adipogenic bacteria species is anaerobic and/or aerobic and wherein the bacteria species is attributable to the strain Actinobacteria, Bacteroidetes, Firmicutes or Proteobacteria.

11. The bacteriophages according to claim 7 for use in medication to change the intestinal flora, in medication for achieving and/or maintaining weight reduction, in medication for preventing weight gain and/or in medication for treating overweight, obesity, a metabolic disorder, cardiovascular disease, a bacterial infection, a metabolic disease, metabolic syndrome and/or cancer.

12. The bacteriophages according to claim 7 for use in treating overweight, obesity, metabolic disorder, cardiovascular disease, bacterial infection, metabolic disease, metabolic syndrome and/or cancer.

13. The bacteriophages according to claim 7 for use in a therapeutic method or in a non-therapeutic method to change the composition of the intestinal flora, in a therapeutic method and/or in a non-therapeutic method for achieving and/or maintaining weight loss, in a therapeutic method or in a non-therapeutic method for preventing weight gain and/or in a therapeutic method and/or in a non-therapeutic method for treating overweight, obesity, metabolic disorder, cardiovascular disease, bacterial infection, metabolic disease, metabolic syndrome and/or cancer.

14. The bacteriophages and/or pharmaceutical composition according to claim 11, wherein the bacterial infection is a bacterial infection of the airways, the teeth, the mouth, the jaw, the eye, the musculoskeletal system, the blood, the gastrointestinal tract, the skin, the cardiovascular system, the hormonal balance, the psyche, the immune system, the nervous system, the metabolism, wounds and/or the urogenital tract.

15. A pharmaceutical composition comprising bacteriophages according to claim 7 and at least one other component for use in medication to change the intestinal flora, in medication for achieving and/or maintaining weight reduction, in medication for preventing weight gain and/or in medication for treating overweight, obesity, a metabolic disorder, cardiovascular disease, a bacterial infection, a metabolic disease, metabolic syndrome and/or cancer.

16. A pharmaceutical composition comprising bacteriophages according to claim 7 and at least one other component for use in treating overweight, obesity, metabolic disorder, cardiovascular disease, bacterial infection, metabolic disease, metabolic syndrome and/or cancer.

17. A pharmaceutical composition comprising bacteriophages according claim 7 and at least one other component for use in a therapeutic method or in a non-therapeutic method to change the composition of the intestinal flora, in a therapeutic method and/or in a non-therapeutic method for achieving and/or maintaining weight loss, in a therapeutic method or in a non-therapeutic method for preventing weight gain and/or in a therapeutic method and/or in a non-therapeutic method for treating overweight, obesity, metabolic disorder, cardiovascular disease, bacterial infection, metabolic disease, metabolic syndrome and/or cancer.
